(19) United States
(12) Patent Application Publication
(10) Pub. No.: US 2025/0082911 A1
(43) Pub. Date: Mar. 13, 2025

(54) HOURGLASS BALLOON-IN-BALLOON SYSTEM

(71) Applicant: BOSTON SCIENTIFIC SCIMED, INC., MAPLE GROVE, MN (US)

(72) Inventors: Brian Fahy, Galway (IE); Jeffrey Steven Fuller, Brooklyn Park, MN (US); Andrew John Mitterholzer, Eden Prairie, MN (US); Soo-Young Yoon, Maple Grove, MN (US)

(73) Assignee: BOSTON SCIENTIFIC SCIMED, INC., MAPLE GROVE, MN (US)

(21) Appl. No.: 18/883,209

(22) Filed: Sep. 12, 2024

Related U.S. Application Data
(60) Provisional application No. 63/582,336, filed on Sep. 13, 2023.

(51) Int. Cl. A61M 25/10

(52) U.S. Cl. CPC A61M 25/1011; A61M 25/1002; A61M 25/10184

(57) ABSTRACT

A balloon system for balloon aortic valvuloplasty includes a delivery catheter, an outer expandable member coupled to the delivery catheter, and an inner balloon disposed within the outer expandable member. The outer expandable member has a first length, and a preset hourglass shape defined by first and second bulbous portions separated by a waist region. The inner balloon is non-compliant and has a second length shorter than the first length.

### CROSS-REFERENCE TO RELATED APPLICATIONS

This application claims the benefit of priority of U.S. Provisional Application No. 63/582,336 filed Sep. 13, 2023, the entire disclosure of which is hereby incorporated by reference.

### TECHNICAL FIELD

The disclosure pertains to medical devices and more particularly to devices for balloon aortic valvuloplasty (BAV), and methods for using such medical devices.

### BACKGROUND

A wide variety of medical devices have been developed for medical use including, for example, medical devices utilized to open a patient's native aortic valve. A BAV procedure is a minimally invasive procedures used to treat aortic stenosis, a narrowing of the aortic valve. An artificial valve may be implanted after a BAV procedure. Transcatheter aortic valve replacement (TAVR), also known as transcatheter aortic valve implantation (TAVI), is another procedure to treat aortic stenosis. Of the known medical devices and methods for treating aortic stenosis, each has certain advantages and disadvantages. There is an ongoing need to provide alternative medical devices as well as alternative methods for manufacturing and using the medical devices.

### SUMMARY

This disclosure provides design, material, manufacturing method, and use alternatives for medical devices. An example balloon system for balloon aortic valvuloplasty includes a delivery catheter, an outer expandable member coupled to the delivery catheter, the outer expandable member having a first length, and having a preset hourglass shape defined by first and second bulbous portions separated by a waist region, and an inner balloon disposed within the outer expandable member, the inner balloon being non-compliant and having a second length shorter than the first length.

Alternatively or additionally to the embodiment above, the outer expandable member is an outer compliant balloon, wherein the inner balloon is disposed within the waist region of the outer compliant balloon.

Alternatively or additionally to any of the embodiments above, the outer balloon and the inner balloon are independently inflatable.

Alternatively or additionally to any of the embodiments above, the inner balloon is configured to expand the waist region of the outer balloon.

Alternatively or additionally to any of the embodiments above, the waist region of the outer compliant balloon has a textured outer surface.

Alternatively or additionally to any of the embodiments above, the textured outer surface includes a plurality of outwardly extending circumferential ribs.

Alternatively or additionally to any of the embodiments above, the textured outer surface includes dimples, a roughened texture, or crosshatched ribbing.

Alternatively or additionally to any of the embodiments above, the delivery catheter includes a first inflation lumen in fluid communication with the outer balloon and a second inflation lumen in fluid communication with the inner balloon.

Alternatively or additionally to any of the embodiments above, the delivery catheter includes a single inflation lumen in fluid communication with both the outer balloon and the inner balloon, and a diversion mechanism configured to allow inflation of both the outer balloon and the inner balloon to a first pressure, and then to selectively inflate only the inner balloon to a second, higher pressure.

Alternatively or additionally to any of the embodiments above, the outer balloon is non-occluding and has a preset flat hourglass shape configured to allow blood to pass by when the outer balloon is disposed within a patient's aortic valve and is inflated.

Alternatively or additionally to any of the embodiments above, the first and second bulbous portions of the outer expandable member have a smaller diameter than diameters of a patient's ventricle and aorta and larger than a diameter of the patient's native aortic valve.

Alternatively or additionally to any of the embodiments above, the first length is 40 mm to 60 mm, and the second length is 10 mm to 20 mm.

Alternatively or additionally to any of the embodiments above, opposing ends of the outer expandable member are fixed to the delivery catheter and the first and second bulbous portions are tapered down to their ends.

Alternatively or additionally to any of the embodiments above, the outer expandable member is a self-expanding wire frame coupled to the delivery catheter, the self-expanding wire frame including at least one wire having the preset hourglass shape defining the first and second bulbous portions and the waist region, wherein the inner balloon is disposed within the waist region.

Alternatively or additionally to any of the embodiments above, the self-expanding wire frame includes first and second wires each having first and second ends fixed to the delivery catheter, where the first and second wires cross one another to define the waist region between the first and second ends.

Alternatively or additionally to any of the embodiments above, the self-expanding wire frame includes only a single wire having first and second ends fixed to the delivery catheter, the single wire having a preset spiral shape with radially outwardly extending first and second bulbous portions and the single wire crossing the delivery catheter to define the waist region.

Another example balloon system for balloon aortic valvuloplasty includes a delivery catheter, an outer balloon coupled to the delivery catheter, the outer balloon being compliant, having a first length, and having a preset hourglass shape defined by first and second bulbous portions separated by a waist region, the first and second bulbous portions having first and second radial diameters at their widest points, respectively, and the waist region having a third radial diameter smaller than the first and second radial diameters, the first bulbous portion defining a first end of the outer balloon and the second bulbous portion defining a second end of the outer balloon, wherein the first and second ends taper from the first and second radial diameters down to the first and second ends of the outer balloon which are fixed to the delivery catheter, and an inner balloon disposed within the outer balloon, the inner balloon being non-compliant and having a second length shorter than the first length, the inner balloon being configured to expand the waist region of the outer balloon.

Alternatively or additionally to the embodiment above, the first and second bulbous portions of the outer balloon have a smooth outer surface and the waist region has a textured outer surface.

Alternatively or additionally to any of the embodiments above, the first length is 40 mm to 60 mm, and the second length is 10 mm to 20 mm.

An example method for balloon aortic valvuloplasty includes inserting a distal end of a delivery catheter through a patient's aortic valve, the delivery catheter including an outer expandable member coupled adjacent the distal end of the delivery catheter, the outer expandable member having a first length and having a preset hourglass shape defined by distal and proximal bulbous portions separated by a waist region having a radial diameter smaller than radial diameters of the distal and proximal bulbous portions (when expanded), and an inner balloon disposed within the outer expandable member, the inner balloon being non-compliant and having a second length shorter than the first length, expanding the outer expandable member with the distal bulbous portion disposed distal of the patient's aortic valve, the proximal bulbous portion disposed proximal of the patient's aortic valve, and the waist region extending across the patient's aortic valve, after expanding the outer expandable member, then expanding the inner balloon to expand the waist region and open the patient's aortic valve, and collapsing the inner balloon and the outer expandable member and removing the delivery catheter from the patient.

The above summary of some embodiments, aspects, and/or examples is not intended to describe each embodiment or every implementation of the present disclosure. The figures and the detailed description which follows more particularly exemplify these embodiments.

### BRIEF DESCRIPTION OF THE DRAWINGS

The disclosure may be more completely understood in consideration of the following detailed description of various embodiments in connection with the accompanying drawings, in which:

FIGS. 1A-1D illustrate a prior art balloon in a BAV procedure;

FIGS. 2A and 2B illustrate an example balloon-in-balloon system in a BAV procedure;

FIGS. 3A-3G illustrate other example balloon-in-balloon systems in a BAV procedure with various inflation mechanisms;

FIGS. 4A-4D illustrate surface textures on the waist region of an hourglass balloon;

FIGS. 5A and 5B illustrate a flat hourglass balloon-in-balloon system in front and side views, respectively;

FIGS. 5C-5F illustrate the flat hourglass balloon-in-balloon system of FIGS. 5A and 5B in a BAV procedure;

FIGS. 6A and 6B illustrate a self-expanding double wire frame and inner balloon system in a BAV procedure; and

FIGS. 7A and 7B illustrate a single wire frame and inner balloon system.

While aspects of the disclosure are amenable to various modifications and alternative forms, specifics thereof have been shown by way of example in the drawings and will be described in detail. It should be understood, however, that the intention is not to limit aspects of the disclosure to the particular embodiments described. On the contrary, the intention is to cover all modifications, equivalents, and alternatives falling within the spirit and scope of the disclosure.

### DETAILED DESCRIPTION

For the following defined terms, these definitions shall be applied, unless a different definition is given in the claims or elsewhere in this specification.

All numeric values are herein assumed to be modified by the term “about,” whether or not explicitly indicated. The term “about”, in the context of numeric values, generally refers to a range of numbers that one of skill in the art would consider equivalent to the recited value (e.g., having the same function or result). In many instances, the term “about” may include numbers that are rounded to the nearest significant figure. Other uses of the term “about” (e.g., in a context other than numeric values) may be assumed to have their ordinary and customary definition(s), as understood from and consistent with the context of the specification, unless otherwise specified.

The recitation of numerical ranges by endpoints includes all numbers within that range, including the endpoints (e.g., 1 to 5 includes 1, 1.5, 2, 2.75, 3, 3.80, 4, and 5). Although some suitable dimensions, ranges, and/or values pertaining to various components, features and/or specifications are disclosed, one of skill in the art, incited by the present disclosure, would understand desired dimensions, ranges, and/or values may deviate from those expressly disclosed.

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise. It is to be noted that in order to facilitate understanding, certain features of the disclosure may be described in the singular, even though those features may be plural or recurring within the disclosed embodiment(s). Each instance of the features may include and/or be encompassed by the singular disclosure(s), unless expressly stated to the contrary. For simplicity and clarity purposes, not all elements of the disclosure are necessarily shown in each figure or discussed in detail below. However, it will be understood that the following discussion may apply equally to any and/or all of the components for which there are more than one, unless explicitly stated to the contrary. Additionally, not all instances of some elements or features may be shown in each figure for clarity.

Relative terms such as “proximal”, “distal”, “advance”, “withdraw”, variants thereof, and the like, may be generally considered with respect to the positioning, direction, and/or operation of various elements relative to a user/operator/manipulator of the device, wherein “proximal” and “withdraw” indicate or refer to closer to or toward the user and “distal” and “advance” indicate or refer to farther from or away from the user. In some instances, the terms “proximal” and “distal” may be arbitrarily assigned in an effort to facilitate understanding of the disclosure, and such instances will be readily apparent to the skilled artisan. Other relative terms, such as “upstream”, “downstream”, “inflow”, and “outflow” refer to a direction of fluid flow within a lumen, such as a body lumen, a blood vessel, or within a device.

The term “extent” may be understood to mean a greatest measurement of a stated or identified dimension, unless the extent or dimension in question is preceded by or identified as a “minimum”, which may be understood to mean a smallest measurement of the stated or identified dimension. For example, “outer extent” may be understood to mean a maximum outer dimension, “radial extent” may be understood to mean a maximum radial dimension, “longitudinal extent” may be understood to mean a maximum longitudinal dimension, etc. Each instance of an “extent” may be different (e.g., axial, longitudinal, lateral, radial, circumferential, etc.) and will be apparent to the skilled person from the context of the individual usage. Generally, an “extent” may be considered a greatest possible dimension measured according to the intended usage, while a “minimum extent” may be considered a smallest possible dimension measured according to the intended usage. In some instances, an “extent” may generally be measured orthogonally within a plane and/or cross-section, but may be, as will be apparent from the particular context, measured differently-such as, but not limited to, angularly, radially, circumferentially (e.g., along an arc), etc. Additionally, the term “substantially” when used in reference to two dimensions being “substantially the same” shall generally refer to a difference of less than or equal to 5%.

The terms “monolithic” and “unitary” shall generally refer to an element or elements made from or consisting of a single structure or base unit/element. A monolithic and/or unitary element shall exclude structure and/or features made by assembling or otherwise joining multiple discrete elements together.

It is noted that references in the specification to “an embodiment”, “some embodiments”, “other embodiments”, etc., indicate that the embodiment(s) described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it would be within the knowledge of one skilled in the art to affect the particular feature, structure, or characteristic in connection with other embodiments, whether or not explicitly described, unless clearly stated to the contrary. That is, the various individual elements described below, even if not explicitly shown in a particular combination, are nevertheless contemplated as being combinable or arrangeable with each other to form other additional embodiments or to complement and/or enrich the described embodiment(s), as would be understood by one of ordinary skill in the art.

For the purpose of clarity, certain identifying numerical nomenclature (e.g., first, second, third, fourth, etc.) may be used throughout the description and/or claims to name and/or differentiate between various described and/or claimed features. It is to be understood that the numerical nomenclature is not intended to be limiting and is exemplary only. In some embodiments, alterations of and deviations from previously-used numerical nomenclature may be made in the interest of brevity and clarity. That is, a feature identified as a “first” element may later be referred to as a “second” element, a “third” element, etc. or may be omitted entirely, and/or a different feature may be referred to as the “first” element. The meaning and/or designation in each instance will be apparent to the skilled practitioner.

The following description should be read with reference to the drawings, which are not necessarily to scale, wherein similar elements in different drawings are numbered the same. The detailed description and drawings are intended to illustrate but not limit the disclosure. Those skilled in the art will recognize that the various elements described and/or shown may be arranged in various combinations and configurations without departing from the scope of the disclosure. The detailed description and drawings illustrate example embodiments of the disclosure. However, in the interest of clarity and ease of understanding, while every feature and/or element may not be shown in each drawing, the feature(s) and/or element(s) may be understood to be present regardless, unless otherwise specified.

Balloon Aortic Valvuloplasty (BAV) is a minimally invasive procedure that mechanically dilates the aortic valve leaflets in patients with severe aortic stenosis. It is most often used in patients who are not suited for valve replacement procedure, patients who require bridging treatment to transcatheter aortic valve replacement (TAVR), cases that require pre-dilation of valves with high calcific load prior to TAVR, and post dilation of prosthetic valves.

BAV and TAVR procedures may introduce a risk of pacemaker dependency due to disruption of the atrioventricular (AV) conduction system. The risk of pacemaker dependency after transcatheter aortic valve replacement may be influenced by patient—and procedure-specific factors, such as the membranous septum (MS) length and inflation pressure and location of balloon during BAV or TAVR. Given that the (AV) bundle runs along the lower border of the MS in the vast majority of subjects, and that the MS lies close to the aortic annulus, balloon inflation pressure on the AV bundle during expansion of the aortic valve, and the frame of a low-implanted transcatheter heart valve may permanently injure the branches emerging on the endocardial surface between the MS and the muscular septum. Additionally, positioning replacement valve frames too low in the annulus have been shown to increase instances of pacemaker dependency.

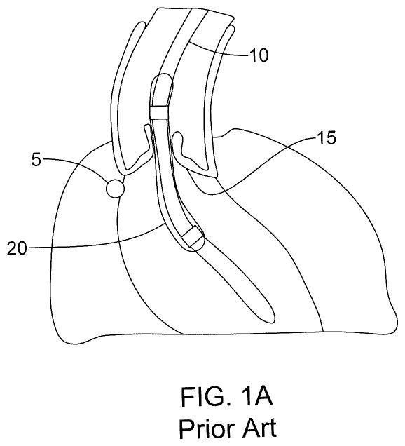

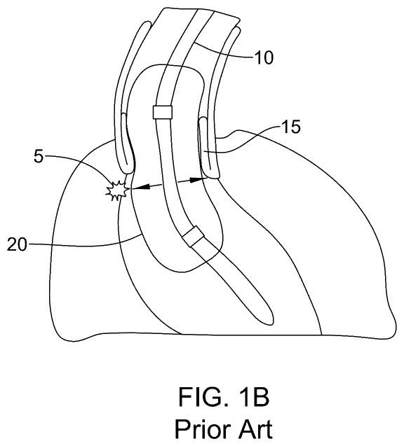

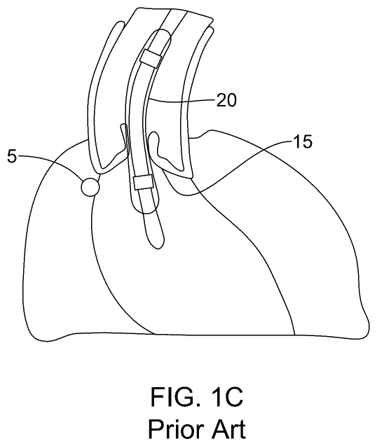

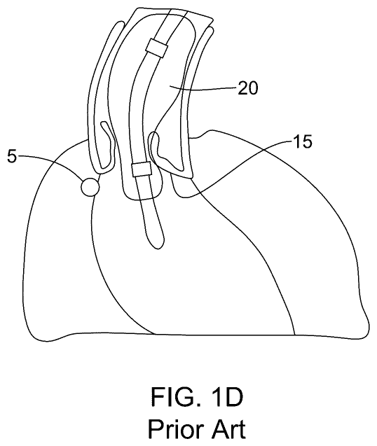

FIGS. 1A-1D illustrate disadvantages of prior art cylindrical balloons used for BAV. FIG. 1A shows the location of the AV bundle 5 below the native aortic valve 15 with a delivery catheter 10 and cylindrical balloon 20 inserted through the aortic valve 15. FIG. 1B shows that expansion of the balloon 20 sufficient to fully expand the aortic valve 15 has also impinged on the AF bundle 5, potentially resulting in pacemaker dependency for the patient. FIG. 1C shows inserting the same cylindrical balloon 20 higher within the aortic valve 15 in order to avoid the AV bundle 5. However, upon inflation, while avoiding impinging on the AF bundle 5, the balloon 20 may fail to fully expand the aortic valve 15 and may be pushed upwards by the aortic valve, often called watermelon seeding, as shown in FIG. 1D.

Prior art single hourglass balloons have disadvantages including radial force lost at the balloon waist which is a crucial position for opening the aortic valve, and the bulbous nature of the balloon at the distal end and expansion diameter in the vicinity of the AV bundle increases the risk of disruption to the conduction system, leading to pacemaker dependency. Further, positioning of the prior art cylindrical balloon and single hourglass balloon devices is largely dependent on the radiopacity of the devices used, the quality of the CT imaging equipment, and the skill of the operator.

The balloon-in-balloon system described below avoids or greatly reduces the risk of pacemaker dependency, and may aid and improve positioning and anchoring of BAV and TAVR devices in the target annulus regardless of the quality of the CT equipment used or the skill of the operator.

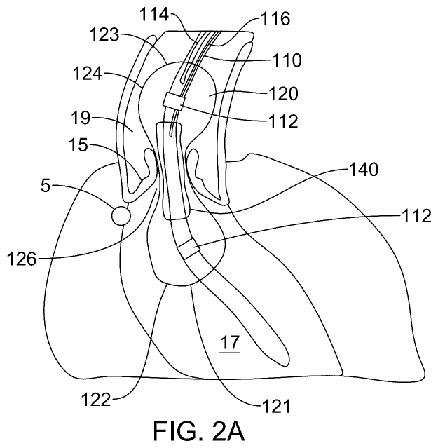

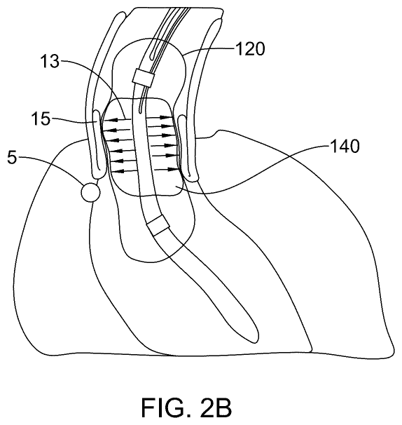

As will be described in greater detail below, FIGS. 2A and 2B illustrate a dual expandable member system for expanding the aortic valve 15 without applying too much pressure on the AV bundle 5. In FIG. 2A the delivery catheter 110 has been inserted through the aortic valve 15. An outer expandable member 120 with a preset hourglass shape is coupled to the delivery catheter 110. The preset hourglass shape of the outer expandable member 120 is defined by a first bulbous portion 122 and a second bulbous portion 124 separated by a waist region 126. The waist region may have a radial diameter that is smaller than the radial diameters of the first and second bulbous portions 122, 124. The outer expandable member 120 has a first length measured longitudinally between a first end 121 and a second end 123. The first and second bulbous portions 122, 124 may each have a round or partially round cross-sectional shape taken transverse to the longitudinal axis of the outer expandable member 120. In some examples, the delivery catheter may include one or more markers 112. The dual expandable member system further includes an inner balloon 140 disposed within the outer expandable member 120. The inner balloon 140 may be non-compliant and have a second length shorter than the first length. In some examples, the first length of the outer expandable member may be 40 mm to 60 mm, and the second length of the inner balloon 140 may be 10 mm to 20 mm. Due to the shorter length of the inner balloon 140, it may be disposed completely inside the outer expandable member 120, with portions of the outer expandable member 120 extending longitudinally beyond the ends of the inner balloon 140, as shown in FIG. 2A. The shorter length of the inner balloon 140 may be sized to expand only the aortic valve 15. The outer expandable member 120 provides a self-locating feature that achieves a high level of placement accuracy in the target annulus. This is achieved by the bulbous portions 122, 124 of the outer expandable member 120, when expanded each having a smaller diameter than the ventricle 17 and aorta 19 but of larger diameter than the native aortic valve 15, causing the device to correct its position on inflation, as shown in FIG. 2A. The inner balloon 140 is then ideally positioned in the aortic valve 15 to inflate and expand the native tissue or valve.

In the embodiment shown in FIGS. 2A and 2B, the dual expandable member system is a balloon-in-balloon system and the outer expandable member 120 is an outer balloon 120, and the inner balloon 140 is disposed within the waist region 126 of the outer balloon 120. The outer balloon 120 may be compliant, and may fill the area of the aortic valve 15 such that the outer balloon 120 is an occluding balloon. In some examples, the outer balloon 120 and the inner balloon 140 may be independently inflatable, with the delivery catheter 110 including a first inflation lumen 114 in fluid communication with the outer balloon 120 and a second inflation lumen 116 in fluid communication with the inner balloon 140.

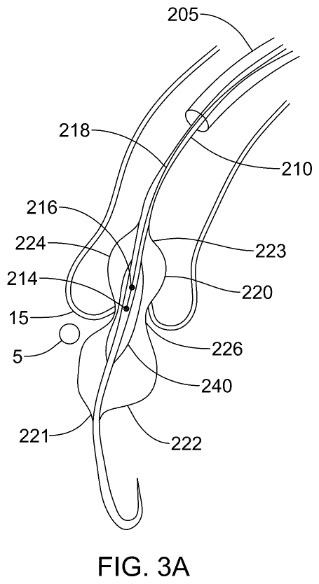

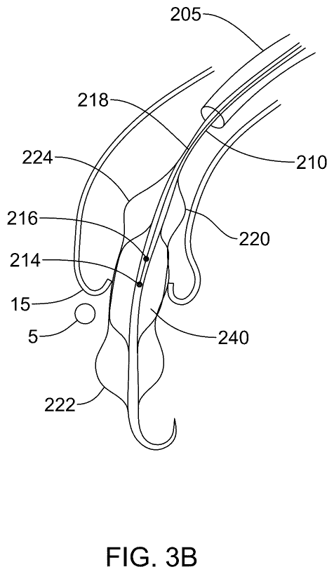

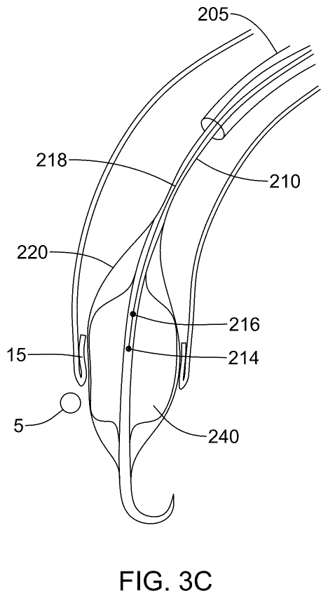

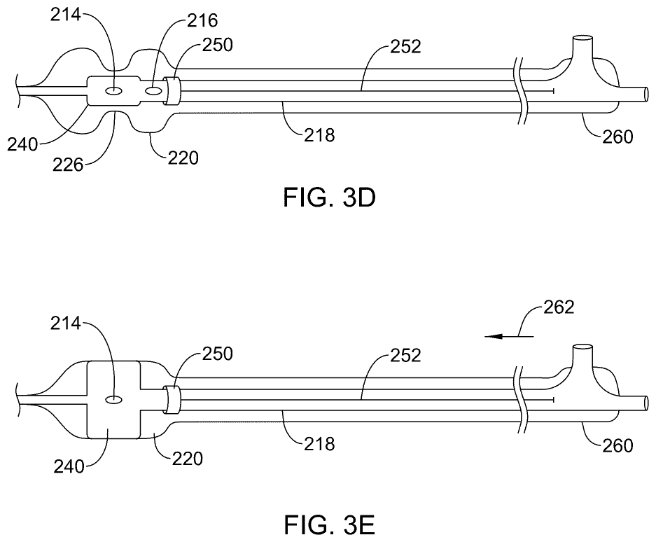

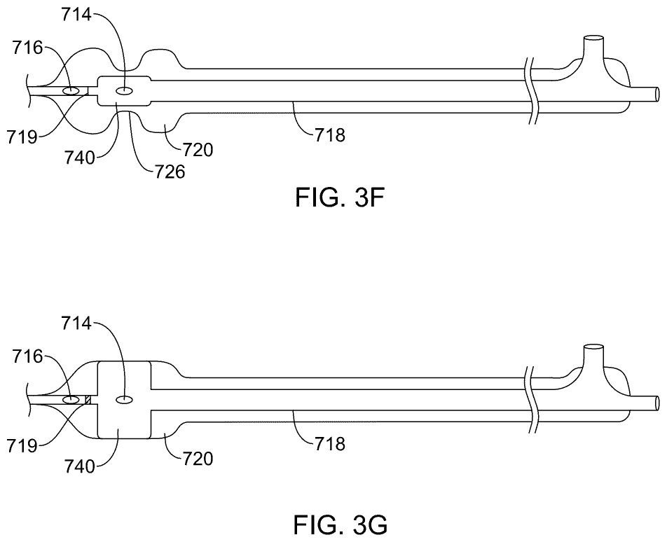

FIGS. 3A-3G illustrate a method of performing a BAV using a balloon-in-balloon system having a delivery sheath 205 and a delivery catheter 210 disposed slidably therein with an outer balloon 220 and an inner balloon 240 coupled to the delivery catheter 210. The outer balloon 220 may have a preset hourglass shape including a first bulbous portion 222 and second bulbous portion 224 with a waist region 226 therebetween. In the embodiment shown in FIGS. 3A-3B, opposing ends of the outer balloon 220 are fixed to the delivery catheter 210 and the first and second bulbous portions 222, 224 are tapered down to their ends. The method includes inserting a distal end of the delivery catheter 210 through the patient's aortic valve 15, and expanding the outer balloon 220 with the first or distal bulbous portion 222 disposed distal of the patient's aortic valve, the second or proximal bulbous portion 224 disposed proximal of the patient's aortic valve, and the waist region 226 extending across the patient's aortic valve, as shown in FIG. 3A.

As seen in FIG. 3A, the first bulbous portion 222 has a first tapered end 221 and the second bulbous portion 224 has a second tapered end 223, such that the bulbous portions 222, 224 have opposing teardrop shapes when inflated. The tapered end 221 of the first bulbous portion 222, which extends below the aortic valve 15, may help avoid impinging on the AV bundle 5 when the outer balloon 220 is fully inflated. In the embodiment shown in FIGS. 3A-3B, the delivery catheter 210 includes a single inflation lumen 218 in fluid communication with both the outer balloon 220 and the inner balloon 240, with a first inflation port 214 into the inner balloon 240 and second inflation port 216 into the outer balloon 220, allowing independent inflation of the outer balloon 220 and inner balloon 240. FIG. 3A illustrates the balloon-in-balloon system after inflation of the outer balloon 220, which may be non-compliant. The preset hourglass shape may aid in positioning the outer balloon in the desired position across the aortic valve 15. The outer balloon 220 may be inflated to 1-2 atm.

Once the operator is satisfied with the positioning of the expanded outer balloon 220, the method may include at least partially inflating the inner balloon 240, expanding the waist region 226 of the outer balloon 220, as shown in FIG. 3B. As shown in FIG. 3C, fully expanding the inner balloon 240 fully expands the waist region 226 of the outer balloon 220 and fully opens the patient's aortic valve 15. The hourglass shape of the outer balloon 220 provides the shorter inner balloon 240 with an ideal position within the aortic valve 15, avoiding any disruption to the AV bundle 5 as the inner balloon 240 is fully expanded to fully open the aortic valve 15. The inner balloon 240 may be fully inflated to 4-5 atm. The inner balloon 240 and the outer balloon 220 may then be collapsed and removed by removing the delivery catheter from the patient.

FIGS. 3D-3G illustrate diversion mechanisms allowing the separate and independent inflation of the outer balloon 220, 720 and inner balloon 240, 740 through a single inflation lumen 218, 718. The single inflation lumen includes a first inflation port 214, 714 in fluid communication with the inner balloon 240, 740, and a second inflation port 216, 716 in fluid communication with the outer balloon 220, 720. The first and second inflation ports may be spaced apart axially along the inflation lumen. The hourglass shaped outer balloon 220, 720 may have a much lower inflation pressure than the inner balloon 240, 740, so inflation fluid can be diverted once the outer balloon reaches its recommended pressure, for example 1-2 atm. Once this pressure is reached, flow to the outer balloon 220, 720 may be closed off either manually, as shown in FIGS. 3D and 3E, or by an automatic valve 719, as shown in FIGS. 3F and 3G. Flow of inflation fluid would then be directed only into the inner balloon 240, 740, which would then expand and inflate to 4-5 atm to expand the aortic valve 15. When the procedure is completed, both the outer balloon 220, 720 and the inner balloon 240, 740 may be deflated simultaneously by applying negative pressure to the inflation lumen 218, 718 with a syringe.

As shown in FIGS. 3D and 3E, a manual diversion mechanism may allow inflation fluid passing through the single inflation lumen 218 to inflate both the outer balloon 220 and inner balloons 240 simultaneously to a first pressure (FIG. 3D), and then to selectively inflate only the inner balloon 240 to a higher pressure (FIG. 3E). The diversion mechanism may be an actuatable seal 250. The actuatable seal 250 may be moved axially along the single inflation lumen 218 to selectively cover the second inflation port 216, as illustrated in FIGS. 3D and 3E. The seal 250 may be fixed to a wire 252 extending proximally to a manifold 260. As shown in FIG. 3D, the resting position for the seal 250 may be proximal of the second inflation port 216 such that both the first inflation port 214 and the second inflation port 216 are uncovered. As the outer balloon 220 may have a lower desired inflation pressure, inflation fluid is initially directed to both the outer balloon 220 and inner balloon 240 through the first and second open inflation ports 214, 216. Once the outer balloon 220 and the inner balloon 240 reach the desired pressure for the outer balloon, such as 1-2 atm, the user may push a portion of the manifold 260, as indicated by arrow 262, to move the wire 252 and the seal 250 distally over the second inflation port 216, as shown in FIG. 3E. With the second inflation port 216 sealed, pressure and shape of the outer balloon 220 is maintained, and the user may continue directing inflation fluid through the inflation lumen 218 to inflate the inner balloon 240 to its higher desired pressure, such as 5-6 atm. In some examples, the seal 250 may extend circumferentially around the single inflation lumen 218. The seal 250 may be a flexible band configured to form a seal against the outer wall of the inflation lumen 218.

An automatic diversion mechanism is shown in FIGS. 3F and 3G. The automatic diversion mechanism may allow inflation fluid passing through the single inflation lumen 718 to inflate both the outer balloon 720 and inner balloon 740 simultaneously to a first pressure (FIG. 3F), and then to selectively inflate only the inner balloon 740 to a higher pressure (FIG. 3G). The automatic diversion mechanism may be a pressure activated seal 719 disposed distal of the inner balloon 740. In this embodiment, the single inflation lumen 718 delivers inflation fluid simultaneously to both the inner balloon 740 and the outer balloon 720 through the first inflation port 714 and second inflation port 716, respectively, until the outer balloon 720 reaches its desired inflation pressure, such as 1-2 atm. At this point, the pressure activated seal 719 will seal off the inflation lumen 718, preventing inflation fluid from entering the outer balloon 720. Continued inflation further inflates the inner balloon 740 until it reaches its desired inflation pressure, such as 5-6 atm. The pressure activated seal 719 will seal off the inflation lumen 718 distal of the inner balloon 740 until the pressure in the inflation lumen 718 drops below the seal's activation pressure, which may be 2 atm. The pressure activated seal 719 may act as a safety mechanism, preventing the outer balloon 720 from inflating to a pressure that could lead to overexpansion or rupture.

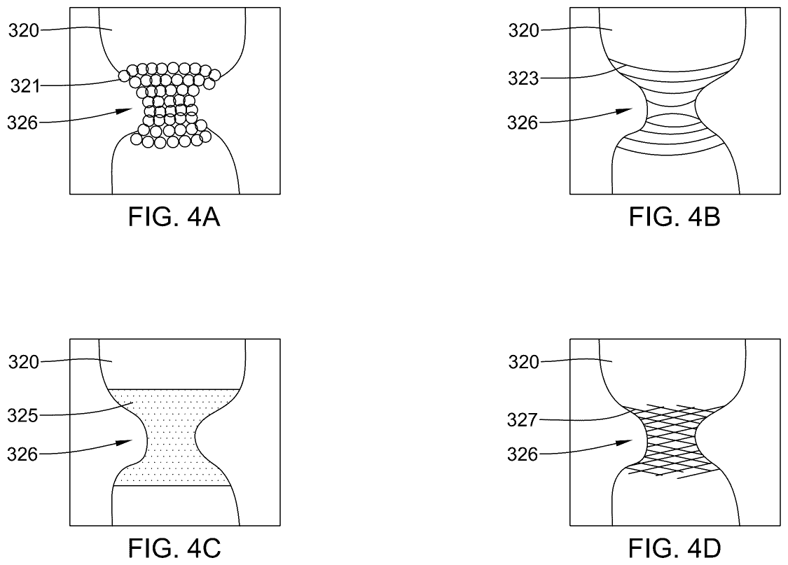

In some examples, any of the above described outer balloons 120, 220, 720 may have a textured outer surface over a portion of the balloon, such as in the waist region 126, 226. FIGS. 4A-4D illustrate part of an hourglass shaped outer balloon 320 having a waist region 326 with a textured outer surface. The textured outer surface may aid in securing the outer balloon 320 within the aortic valve. FIG. 4A illustrates a bubbled or dimpled texture in which a plurality of rounded shapes 321 extend radially outward from the outer surface of the outer balloon 320. The rounded shapes 321 may extend over only the waist region 326 or they may extend partially onto one or both of the bulbous portions. FIG. 4B illustrates a plurality of circumferential ribs 323 extending radially outward from the outer surface of the outer balloon 320. The circumferential ribs 323 may provide increased axial securement within the aortic valve. FIG. 4C illustrates the outer balloon 320 with a roughened texture 325 on the waist region. The roughened texture 325 may include an uneven radially raised bumps. FIG. 4D illustrates a pattern of cross-hatched ribs 327 on the waist region of the outer balloon 320. The cross-hatched ribs 327 may be radially outwardly extending ribs present in a regular pattern or in a random arrangement. In some examples, the first and second bulbous portions of the outer balloon 320 may have a smooth outer surface.

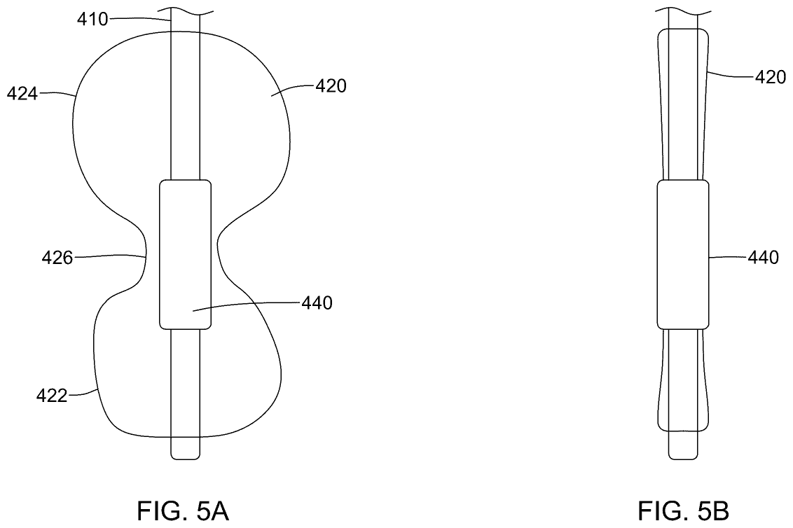

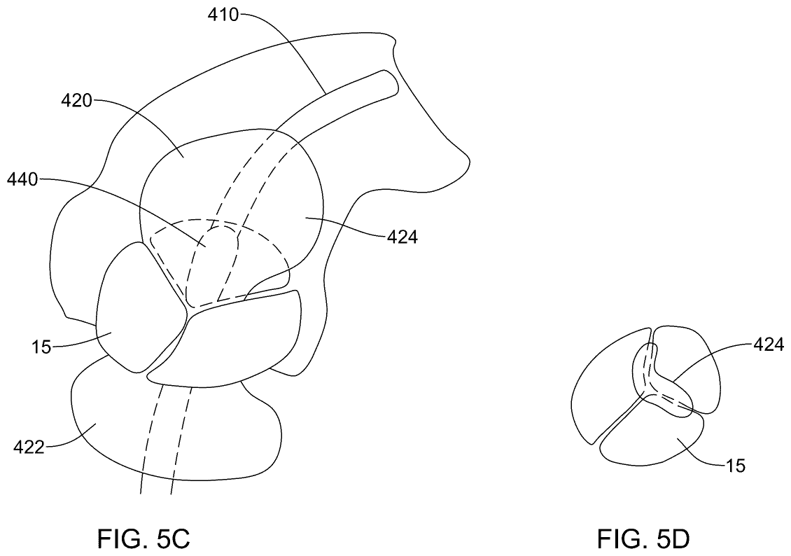

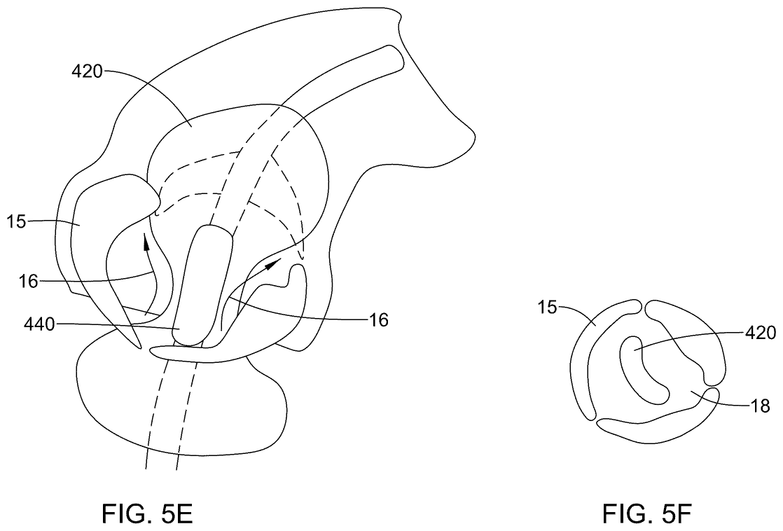

Instead of an outer balloon with a round transverse cross-section as discussed above, in some examples the outer balloon 420 may be non-occluding and have a preset flat hourglass shape as illustrated in FIGS. 5A-5F. The flat shape is configured to allow blood to pass by when the outer balloon 420 is disposed within a patient's aortic valve and is inflated. FIG. 5A shows a front view of the hourglass shaped outer balloon 420 with inner balloon 440 on a delivery catheter 410. The first and second bulbous portions 422, 424 are separated by a waist region 426 with the inner balloon 440 disposed within the waist region 426. FIG. 5B shows a side view of the same outer balloon 420, inner balloon 440, and delivery catheter 410, illustrating the flat dimension. The outer balloon 420 and inner balloon 440 may be the same as the balloons described above with the only difference being the flat hourglass shape. FIGS. 5C-5F illustrate the flat balloon system disposed within the aortic valve. In FIG. 5C, the outer balloon 420 has been expanded with the first and second bulbous portions 422, 424 providing positioning of the outer balloon within the aortic valve, while the inner balloon 440 remains unexpanded. As shown in the top down view in FIG. 5D, the second bulbous portion 224 of the flat outer balloon 420 is disposed along the interface between adjacent valve leaflets, without significantly expanding the aortic valve 15. FIG. 5E shows the flat outer balloon 420 allowing blood flow, indicated by arrows 16, around the outer balloon 420 when the aortic valve opens. FIG. 5F is a top down view of FIG. 5E, also showing the space 18 for blood to flow around the outer balloon 420 when the aortic valve 15 opens. This embodiment allows for blood to flow around the expanded outer balloon 420 during positioning. Occlusion only occurs when the inner balloon 440 is expanded to force the aortic valve open.

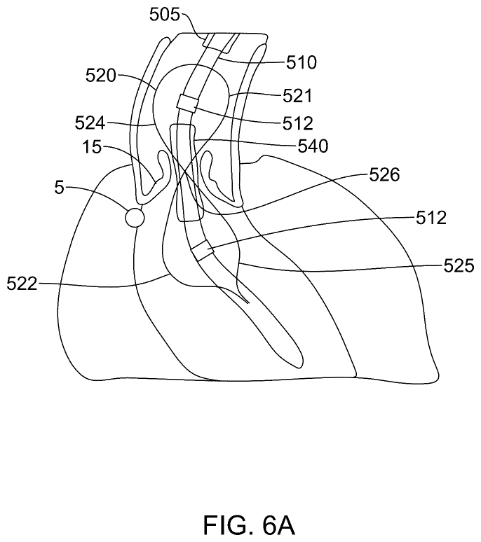

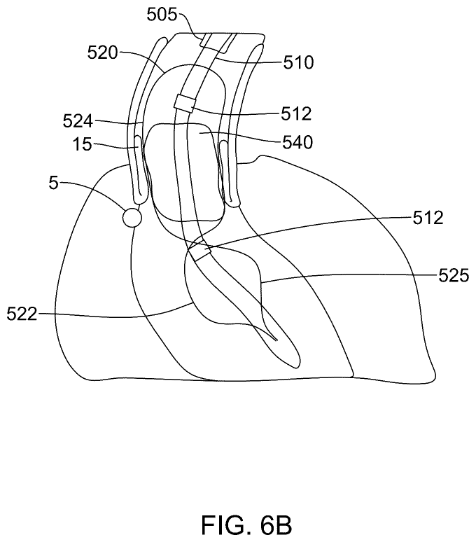

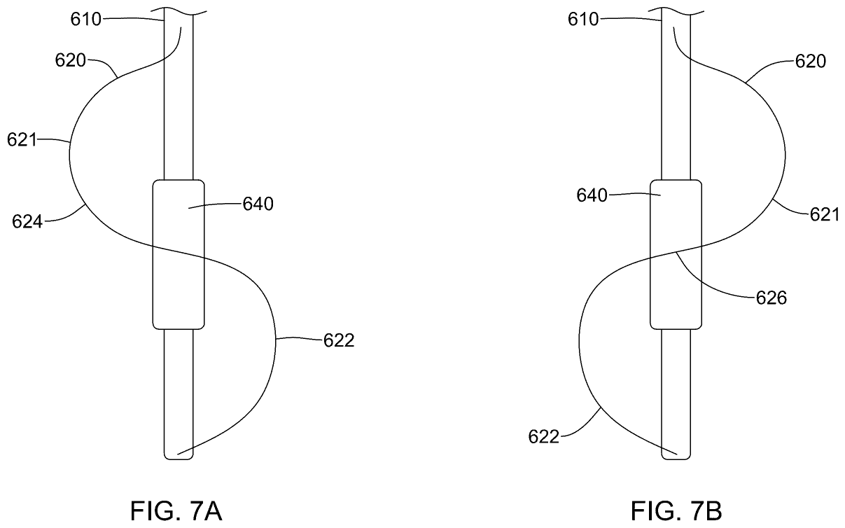

FIGS. 6A-7B illustrate further embodiments in which the outer expandable member is a self-expanding wire frame coupled to the delivery catheter, where the self-expanding wire frame includes at least one wire having the preset hourglass shape defining the first and second bulbous portions and the waist region. The inner balloon may be disposed within the waist region of the self-expanding wire frame and may be the same as the non-compliant inner balloons described above.

In the embodiment shown in FIGS. 6A and 6B, the self-expanding wire frame 520 includes a first wire 521 and a second wire 525 each having first and second ends fixed to the delivery catheter 510. The first and second wires 521, 525 cross one another to define the waist region 526 and first and second bulbous portions 522, 524. The inner balloon 540 may extend across the waist region 526. When the assembly is inserted through the aortic valve 15 and the self-expanding wire frame 520 is moved out of the delivery sheath 505 and allowed to self-expand, the first and second bulbous portions 522, 525 hold the inner balloon 540 in position across the aortic valve 15. The first and second wires 521, 525 may be stiff, collapsible wires that are pre-shaped into the hourglass shape. The wire configuration defining the outer expandable member allows for blood flow through the aortic valve 15 during insertion, positioning, and expansion of the self-expanding wire frame 520. One or more markers 512 may be disposed on the delivery catheter 510 and may aid the user in positioning the self-expanding wire frame 520 relative to the aortic valve 15. FIG. 6B shows the system of FIG. 6A with the inner balloon 540 fully expanded, moving aside the first and second wires 521, 525 and fully opening the aortic valve 15. As shown in FIG. 6B, the wire structure of the self-expanding wire frame and short inner balloon 540 avoids interfering with the AV bundle 5 when the inner balloon 540 is fully expanded.

FIGS. 7A and 7B illustrate an embodiment of self-expanding wire frame 620 including only a single wire 621 having first and second ends fixed to the delivery catheter 610. The single wire 621 may have a preset spiral shape with radially outwardly extending first and second bulbous portions 622, 624 and crosses the delivery catheter 610 and inner balloon 640 to define the waist region 626. The inner balloon 640 may extend across the waist region 626. The single wire 621 may spiral around the catheter 610, forming an overall rounded configuration when viewed down the length of the delivery catheter 610.

The materials that can be used for the various components of the system (and/or other elements disclosed herein) and the various components thereof disclosed herein may include those commonly associated with medical devices and/or systems. For simplicity purposes, the following discussion refers to the system. However, this is not intended to limit the devices and methods described herein, as the discussion may be applied to other elements, members, components, or devices disclosed herein, such as, but not limited to, the occlusive implant, the delivery sheath, the core wire, the expandable framework, the occlusive element, the capsule, the elongate fingers, the elongate strand, etc. and/or elements or components thereof.

In some embodiments, the system and/or components thereof may be made from a metal, metal alloy, polymer, a metal-polymer composite, ceramics, combinations thereof, and the like, or other suitable material.

Some examples of suitable polymers may include polytetrafluoroethylene (PTFE), ethylene tetrafluoroethylene (ETFE), fluorinated ethylene propylene (FEP), polyoxymethylene (POM; for example, DELRIN®), polyether block ester, polyurethane, polypropylene (PP), polyvinylchloride (PVC), polyether-ester (for example, ARNITEL®), ether or ester based copolymers (for example, butylene/poly(alkylene ether) phthalate and/or other polyester elastomers such as HYTREL®), polyamide (for example, DURETHAN® or CRISTAMID®), elastomeric polyamides, block polyamide/ethers, polyether block amide (PEBA; for example, PEBAX®), ethylene vinyl acetate copolymers (EVA), silicones, polyethylene (PE), MARLEX® high-density polyethylene, MARLEX® low-density polyethylene, linear low density polyethylene (for example, REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID®), perfluoro (propyl vinyl ether) (PFA), ethylene vinyl alcohol, polyolefin, polystyrene, epoxy, polyvinylidene chloride (PVdC), poly(styrene-b-isobutylene-b-styrene) (for example, SIBS and/or SIBS 50A), polycarbonates, polyurethane silicone copolymers (for example, Elast-Eon® or ChronoSil®), biocompatible polymers, other suitable materials, or mixtures, combinations, copolymers thereof, polymer/metal composites, and the like. In some embodiments, the system and/or components thereof can be blended with a liquid crystal polymer (LCP). For example, the mixture can contain up to about 6 percent LCP.

Some examples of suitable metals and metal alloys include stainless steel, such as 304 and/or 316 stainless steel and/or variations thereof; mild steel; nickel-titanium alloy such as linear-elastic and/or super-elastic nitinol; other nickel alloys such as nickel-chromium-molybdenum alloys (e.g., UNS: N06625 such as INCONEL® 625, UNS: N06022 such as HASTELLOY® C-22®, UNS: N10276 such as HASTELLOY® C276®, other HASTELLOY® alloys, and the like), nickel-copper alloys (e.g., UNS: N04400 such as MONEL® 400, NICKELVAC® 400, NICORROS® 400, and the like), nickel-cobalt-chromium-molybdenum alloys (e.g., UNS: R30035 such as MP35-N® and the like), nickel-molybdenum alloys (e.g., UNS: N10665 such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R30003 such as ELGILOY®, PHYNOX®, and the like); platinum enriched stainless steel; titanium; platinum; palladium; gold; combinations thereof; or any other suitable material.

In at least some embodiments, portions or all of the system and/or components thereof may also be doped with, made of, or otherwise include a radiopaque material. Radiopaque materials are understood to be materials capable of producing a relatively bright image on a fluoroscopy screen or another imaging technique (e.g., ultrasound, etc.) during a medical procedure. This relatively bright image aids the user of the system in determining its location. Some examples of radiopaque materials can include, but are not limited to, gold, platinum, palladium, tantalum, tungsten alloy, polymer material loaded with a radiopaque filler, and the like. Additionally, other radiopaque marker bands and/or coils may also be incorporated into the design of the system to achieve the same result.

In some embodiments, the system and/or components thereof may include a fabric material. The fabric material may be composed of a biocompatible material, such a polymeric material or biomaterial, adapted to promote tissue ingrowth. In some embodiments, the fabric material may include a bioabsorbable material. Some examples of suitable fabric materials include, but are not limited to, polyethylene glycol (PEG), nylon, polytetrafluoroethylene (PTFE, ePTFE), a polyolefinic material such as a polyethylene, a polypropylene, polyester, polyurethane, and/or blends or combinations thereof.

In some embodiments, the system and/or components thereof may include and/or be formed from a textile material. Some examples of suitable textile materials may include synthetic yarns that may be flat, shaped, twisted, textured, pre-shrunk or un-shrunk. Synthetic biocompatible yarns suitable for use in the present disclosure include, but are not limited to, polyesters, including polyethylene terephthalate (PET) polyesters, polypropylenes, polyethylenes, polyurethanes, polyolefins, polyvinyls, polymethylacetates, polyamides, naphthalene dicarboxylene derivatives, natural silk, and polytetrafluoroethylenes. Moreover, at least one of the synthetic yarns may be a metallic yarn or a glass or ceramic yarn or fiber. Useful metallic yarns include those yarns made from or containing stainless steel, platinum, gold, titanium, tantalum or a Ni—Co—Cr-based alloy. The yarns may further include carbon, glass or ceramic fibers. Desirably, the yarns are made from thermoplastic materials including, but not limited to, polyesters, polypropylenes, polyethylenes, polyurethanes, polynaphthalenes, polytetrafluoroethylenes, and the like. The yarns may be of the multifilament, monofilament, or spun types. The type and denier of the yarn chosen may be selected in a manner which forms a biocompatible and implantable prosthesis and, more particularly, a vascular structure having desirable properties.

In some embodiments, the system and/or components thereof may include and/or be treated with a suitable therapeutic agent. Some examples of suitable therapeutic agents may include anti-thrombogenic agents (such as heparin, heparin derivatives, urokinase, and PPack (dextrophenylalanine proline arginine chloromethyl ketone)); anti-proliferative agents (such as enoxaparin, angiopeptin, monoclonal antibodies capable of blocking smooth muscle cell proliferation, hirudin, and acetylsalicylic acid); anti-inflammatory agents (such as dexamethasone, prednisolone, corticosterone, budesonide, estrogen, sulfasalazine, and mesalamine); antineoplastic/antiproliferative/anti-mitotic agents (such as paclitaxel, 5-fluorouracil, cisplatin, vinblastine, vincristine, epothilones, endostatin, angiostatin and thymidine kinase inhibitors); anesthetic agents (such as lidocaine, bupivacaine, and ropivacaine); anti-coagulants (such as D-Phe-Pro-Arg chloromethyl ketone, an RGD peptide-containing compound, heparin, anti-thrombin compounds, platelet receptor antagonists, anti-thrombin antibodies, anti-platelet receptor antibodies, aspirin, prostaglandin inhibitors, platelet inhibitors, and tick antiplatelet peptides); vascular cell growth promoters (such as growth factor inhibitors, growth factor receptor antagonists, transcriptional activators, and translational promoters); vascular cell growth inhibitors (such as growth factor inhibitors, growth factor receptor antagonists, transcriptional repressors, translational repressors, replication inhibitors, inhibitory antibodies, antibodies directed against growth factors, bifunctional molecules consisting of a growth factor and a cytotoxin, bifunctional molecules consisting of an antibody and a cytotoxin); immunosuppressants (such as the “olimus” family of drugs, rapamycin analogues, macrolide antibiotics, biolimus, everolimus, zotarolimus, temsirolimus, picrolimus, novolimus, myolimus, tacrolimus, sirolimus, pimecrolimus, etc.); cholesterol-lowering agents; vasodilating agents; and agents which interfere with endogenous vasoactive mechanisms.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The scope of the disclosure is, of course, defined in the language in which the appended claims are expressed.

What is claimed is:

1. A balloon system for balloon aortic valvuloplasty, the balloon system comprising:
a delivery catheter;
an outer expandable member coupled to the delivery catheter, the outer expandable member having a first length, and having a preset hourglass shape defined by first and second bulbous portions separated by a waist region; and
an inner balloon disposed within the outer expandable member, the inner balloon being non-compliant and having a second length shorter than the first length.

2. The balloon system of claim 1, wherein the outer expandable member is an outer compliant balloon, wherein the inner balloon is disposed within the waist region of the outer compliant balloon.

3. The balloon system of claim 2, wherein the outer balloon and the inner balloon are independently inflatable.

4. The balloon system of claim 2, wherein the inner balloon is configured to expand the waist region of the outer balloon.

5. The balloon system of claim 2, wherein the waist region of the outer compliant balloon has a textured outer surface.

6. The balloon system of claim 5, wherein the textured outer surface includes a plurality of outwardly extending circumferential ribs.

7. The balloon system of claim 5, wherein the textured outer surface includes dimples, a roughened texture, or crosshatched ribbing.

8. The balloon system of claim 2, wherein the delivery catheter includes a first inflation lumen in fluid communication with the outer balloon and a second inflation lumen in fluid communication with the inner balloon.

9. The balloon system of claim 2, wherein the delivery catheter includes a single inflation lumen in fluid communication with both the outer balloon and the inner balloon, and a diversion mechanism configured to allow inflation of both the outer balloon and the inner balloon to a first pressure, and then to selectively inflate only the inner balloon to a second, higher pressure.

10. The balloon system of claim 2, wherein the outer balloon is non-occluding and has a preset flat hourglass shape configured to allow blood to pass by when the outer balloon is disposed within a patient's aortic valve and is inflated.

11. The balloon system of claim 1, wherein the first and second bulbous portions of the outer expandable member have a smaller diameter than diameters of a patient's ventricle and aorta and larger than a diameter of the patient's native aortic valve.

12. The balloon system of claim 1, wherein the first length is 40 mm to 60 mm, and the second length is 10 mm to 20 mm.

13. The balloon system of claim 1, wherein opposing ends of the outer expandable member are fixed to the delivery catheter and the first and second bulbous portions are tapered down to their ends.

14. The balloon system of claim 1, wherein the outer expandable member is a self-expanding wire frame coupled to the delivery catheter, the self-expanding wire frame including at least one wire having the preset hourglass shape defining the first and second bulbous portions and the waist region, wherein the inner balloon is disposed within the waist region.

15. The balloon system of claim 14, wherein the self-expanding wire frame includes first and second wires each having first and second ends fixed to the delivery catheter, where the first and second wires cross one another to define the waist region between the first and second ends.

16. The balloon system of claim 14, wherein the self-expanding wire frame includes only a single wire having first and second ends fixed to the delivery catheter, the single wire having a preset spiral shape with radially outwardly extending first and second bulbous portions and the single wire crossing the delivery catheter to define the waist region.

17. A balloon system for balloon aortic valvuloplasty, the balloon system comprising:
a delivery catheter;
an outer balloon coupled to the delivery catheter, the outer balloon being compliant, having a first length, and having a preset hourglass shape defined by first and second bulbous portions separated by a waist region, the first and second bulbous portions having first and second radial diameters at their widest points, respectively, and the waist region having a third radial diameter smaller than the first and second radial diameters, the first bulbous portion defining a first end of the outer balloon and the second bulbous portion defining a second end of the outer balloon, wherein the first and second ends taper from the first and second radial diameters down to the first and second ends of the outer balloon which are fixed to the delivery catheter; and
an inner balloon disposed within the outer balloon, the inner balloon being non-compliant and having a second length shorter than the first length, the inner balloon being configured to expand the waist region of the outer balloon.

18. The balloon system of claim 17, wherein the first and second bulbous portions of the outer balloon have a smooth outer surface and the waist region has a textured outer surface.

19. The balloon system of claim 17, wherein the first length is 40 mm to 60 mm, and the second length is 10 mm to 20 mm.

20. A method for balloon aortic valvuloplasty, the method comprising:
inserting a distal end of a delivery catheter through a patient's aortic valve, the delivery catheter including an outer expandable member coupled adjacent the distal end of the delivery catheter, the outer expandable member having a first length and having a preset hourglass shape defined by distal and proximal bulbous portions separated by a waist region having a radial diameter smaller than radial diameters of the distal and proximal bulbous portions (when expanded), and an inner balloon disposed within the outer expandable member, the inner balloon being non-compliant and having a second length shorter than the first length;
expanding the outer expandable member with the distal bulbous portion disposed distal of the patient's aortic valve, the proximal bulbous portion disposed proximal of the patient's aortic valve, and the waist region extending across the patient's aortic valve;
after expanding the outer expandable member, then expanding the inner balloon to expand the waist region and open the patient's aortic valve; and
collapsing the inner balloon and the outer expandable member and removing the delivery catheter from the patient.
